(19) United States
(12) Patent Application Publication
(10) Pub. No.: US 2023/0029475 A1
(43) Pub. Date: Feb. 2, 2023

(54) HYDROGEL THREE-DIMENSIONAL PRINTING

(71) Applicant: HEWLETT-PACKARD DEVELOPMENT COMPANY, L.P., SPRING, TX (US)

(72) Inventors: EMRE HIRO DISCEKICI, SAN DIEGO, CA (US); SHANNON REUBEN WOODRUFF, SAN DIEGO, CA (US); JACOB WRIGHT, SAN DIEGO, CA (US); GRACIELA EMMA NEGRI JIMENEZ, SAN DIEGO, CA (US); CAROLIN FLEISCHMANN, SAN DIEGO, CA (US)

(21) Appl. No.: 17/758,165

(22) PCT Filed: Jan. 30, 2020
(86) PCT No.: PCT/US2020/015771

§ 371 Date: Jun. 29, 2022

(51) Int. Cl. C09D 11/38; C09D 11/106; B33Y 10/00; B33Y 30/00; B33Y 70/00; B29C 64/165

(52) U.S. Cl. CPC C09D 11/38; C09D 11/106; B33Y 10/00; B33Y 30/00; B33Y 70/00; B29C 64/165; B29K 2029/04

(57) ABSTRACT

This disclosure describes hydrogel three-dimensional printing kits, methods of three-dimensional printing hydrogels, and hydrogel three-dimensional printing systems. In one example, a hydrogel three-dimensional printing kit can include a particulate build material and a crosslinking agent. The particulate build material can include from about 90 wt % to 100 wt % of a polyhydroxylated swellable polymer. The crosslinking agent can include water and a crosslinker that is reactive with hydroxyl groups of the polyhydroxylated swellable polymer to crosslink the polyhydroxylated swellable polymer.

### BACKGROUND

Methods of three-dimensional (3D) digital printing, a type of additive manufacturing, have continued to be developed over the last few decades.

However, systems for three-dimensional printing have historically been very expensive, though those expenses have been coming down to more affordable levels recently. Three-dimensional printing technology can shorten the product development cycle by allowing rapid creation of prototype models for reviewing and testing. Unfortunately, the concept has been somewhat limited with respect to commercial production capabilities because the range of materials used in three-dimensional printing is likewise limited. Various materials can be unsuitable for use with certain three-dimensional printing processes. Due to the number of variables involved in three-dimensional printing with new materials, it can be difficult to design three-dimensional printing processes capable of three-dimensional printing with new materials while also providing print accuracy and maintaining the desired material properties in three-dimensional printed objects.

### BRIEF DESCRIPTION OF THE DRAWINGS

FIG. 1 is a schematic view of an example hydrogel three-dimensional printing kit in accordance with examples of the present disclosure.

FIG. 2 is a schematic illustration of a crosslinking reaction of a borate ion with polyvinyl alcohol polymer strands in accordance with examples of the present disclosure.

FIG. 3 is a flowchart of an example method of three-dimensional printing a hydrogel in accordance with examples of the present disclosure.

FIGS. 4A-4C show a schematic view of an example method of three-dimensional printing a hydrogel in accordance with examples of the present disclosure.

FIG. 5 is a schematic view of an example hydrogel three-dimensional printing system in accordance with examples of the present disclosure.

### DETAILED DESCRIPTION

The present disclosure describes hydrogel three-dimensional printing kits, methods of three-dimensional printing hydrogels, and hydrogel three-dimensional printing systems. In one example, a hydrogel three-dimensional printing kit includes a particulate build material and a crosslinking agent. The particulate build material includes from about 90 wt % to 100 wt % of a polyhydroxylated swellable polymer. The crosslinking agent includes water and a crosslinker that is reactive with hydroxyl groups of the polyhydroxylated swellable polymer to crosslink the polyhydroxylated swellable polymer. In some examples, the polyhydroxylated swellable polymer can include polyvinyl alcohol, cellulose, gelatin, alginate, chitosan, poly(2-hydroxyethyl acrylate), poly(2-hydroxyethyl methacrylate), poly(acrylic acid), poly(methacrylic acid), poly(N,N-dimethylacrylamide), poly(N,N-diethylacrylamide), poly(N-isopropylacrylamide), or a combination thereof. In other examples, the polyhydroxylated swellable polymer can be polyvinyl alcohol having a weight average molecular weight from about 1,000 Mw to about 500,000 Mw. In further examples, the crosslinker can be reactive to form hydrogen bonds with the hydroxyl groups of the polyhydroxylated swellable polymer. In certain examples, the crosslinker can include boric acid, citric acid, succinic acid, cationic calcium, cationic barium, or a combination thereof. In other examples, the crosslinker can be present in the crosslinking agent at a concentration from about 0.1 wt % to about 50 wt %.

The present disclosure also describes methods of three-dimensional printing hydrogels. In one example, a method of three-dimensional printing a hydrogel includes: iteratively applying individual layers of a particulate build material, wherein the particulate build material includes from about 90 wt % to 100 wt % of a polyhydroxylated swellable polymer; and, based on a three-dimensional object model, iteratively and selectively applying a crosslinking agent onto the individual layers, wherein the crosslinking agent includes water and a crosslinker. The water swells the polyhydroxylated swellable polymer and the crosslinker reacts with hydroxyl groups of the polyhydroxylated swellable polymer to crosslink the polyhydroxylated swellable polymer, thereby forming a three-dimensional printed hydrogel. In some examples, the particulate build material can be at a temperature from about 0° C. to about 75° C. during three-dimensional printing of the hydrogel. In further examples, the crosslinking agent can be applied onto the individual layers at a contone level so that the individual layers 0include from about 50 wt % to about 95 wt % water based on a total weight of particulate build material and crosslinking agent applied. In other examples, the method can also include removing the three-dimensional printed hydrogel from the powder bed and rinsing the three-dimensional printed hydrogel with water. In certain examples, the polyhydroxylated swellable polymer can include polyvinyl alcohol, cellulose, gelatin, alginate, chitosan, poly(2-hydroxyethyl acrylate), poly(2-hydroxyethyl methacrylate), poly(acrylic acid), poly(methacrylic acid), poly(N,N-dimethylacrylamide), poly(N,N-diethylacrylamide), poly(N-isopropylacrylamide), or a combination thereof. In other examples, the crosslinker can be present in the crosslinking agent in an amount from about 0.1 wt % to about 50 wt %, and the crosslinker can include boric acid, citric acid, succinic acid, cationic calcium, cationic barium, or a combination thereof.

The present disclosure also describes hydrogel three-dimensional printing systems. In one example, a hydrogel three-dimensional printing system includes a particulate build material including from about 90 wt % to 100 wt % of a polyhydroxylated swellable polymer; and a fluid applicator fluidly coupled or coupleable to a crosslinking agent. The fluid applicator is directable to iteratively apply the crosslinking agent to layers of the particulate build material. The crosslinking agent includes water to swell the polyhydroxylated swellable polymer and a crosslinker that is reactive to crosslink hydroxyl groups of the polyhydroxylated swellable polymer. In some examples, the polyhydroxylated swellable polymer can include polyvinyl alcohol, cellulose, gelatin, alginate, chitosan, poly(2-hydroxyethyl acrylate), poly(2-hydroxyethyl methacrylate), poly(acrylic acid), poly(methacrylic acid), poly(N,N-dimethylacrylamide), poly(N,N-diethylacrylamide), poly(N-isopropylacrylamide), or a combination thereof. In further examples, the crosslinker can be present in the crosslinking agent in an amount from about 0.1 wt % to about 50 wt %. The crosslinker can include boric acid, citric acid, succinic acid, cationic calcium, cationic barium, or a combination thereof.

It is noted that when discussing the hydrogel three-dimensional printing kits, methods, and systems herein, these discussions can be considered applicable to one another whether or not they are explicitly discussed in the context of that example. Thus, for example, when discussing a crosslinking agent related to a three-dimensional printing kit, such disclosure is also relevant to and directly supported in the context of methods and systems, vice versa, etc.

It is also understood that terms used herein will take on their ordinary meaning in the relevant technical field unless specified otherwise. In some instances, there are terms defined more specifically throughout the specification or included at the end of the present specification, and thus, these terms have a meaning as described herein.

### Hydrogel Three-Dimensional Printing Kits

The three-dimensional printing kits, methods, and systems described herein can be used to make three-dimensional printed hydrogels.

Hydrogels are a material made up of a network of hydrophilic polymer chains permeated by a relatively large amount of water. The polymer network can maintain its structure while holding the water, thus forming a gel. In various examples, water can make up 10 wt % or more of the hydrogel. In some examples, hydrogels can include water in an amount from about 50 wt % to about 95 wt % or more. The high water content and flexible nature of the polymer network can allow the hydrogel to be flexible. The degree of flexibility of the hydrogel can depend on variables such as the water content of the hydrogel, the properties of the polymer, degree of crosslinking of the polymer, and others.

Hydrogels have many applications in the field of life sciences.

Scaffolds for tissue engineering can be made from hydrogels. The high water content of the hydrogel can provide a suitable environment for living cells. In certain examples, the methods described herein can be performed at temperatures that can be suitable for living cells, such as near normal body temperature. Accordingly, these methods can be used for applications in which living cells may be present during the three-dimensional printing process. Hydrogels can also be used as a medium for cell culture. Additionally, hydrogels can be injectable or implantable and may be used to deliver drugs or help with tissue regeneration. Hydrogels can also be used for a variety of other applications related to life sciences or in other non-related fields.

The hydrogels formed using the three-dimensional printing kits, methods, and systems described herein can be crosslinked. In some examples, the hydrogels can be formed using a layer-by-layer process in which individual layers of polymer particles are crosslinked by applying a crosslinking agent. A layer of dry polymer particles can be spread in a powder bed and then a liquid crosslinking agent can be applied to the powder bed. In certain examples, the crosslinking agent can be applied using a fluid ejector similar to an inkjet printhead. The crosslinking agent can be applied precisely with high resolution to certain areas of the powder bed. The polymer particles that contact the crosslinking agent can become crosslinked together. At the same time, the polymer particles can absorb water from the crosslinking agent. This can cause the crosslinked polymer to swell and become a hydrogel. Additional layers of dry polymer particles can be spread on the powder bed, and additional crosslinking agents can be applied to form more crosslinked layers of hydrogel. The individual layers can have shapes corresponding to layers or slices of a three-dimensional object model. Multiple layers can be formed using this process, and the crosslinking agent can cause the individual layers to crosslink together, forming a continuous hydrogel matrix. In this way, a hydrogel object can be formed having any desired three-dimensional shape. Once the three-dimensional hydrogel object is complete, the object can be removed from the powder bed. Any stray dry polymer particles on the surface of the object can be removed by rinsing, scrubbing, or another method. In certain examples, the polymer powder can be water soluble and therefore easily removed by rinsing the hydrogel object in water.

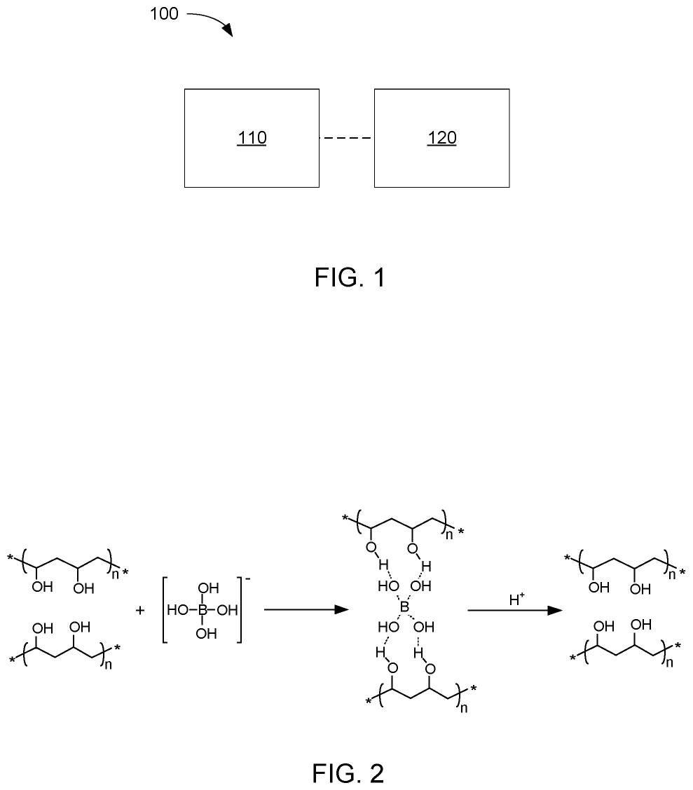

With this description in mind, FIG. 1 shows a schematic view of an example hydrogel three-dimensional printing kit 100. The kit includes a particulate build material 110 and a crosslinking agent 120. The particulate build material includes from about 90 wt % to 100 wt % of a polyhydroxylated swellable polymer. The crosslinking agent includes water and a crosslinker that is reactive with hydroxyl groups of the polyhydroxylated swellable polymer to crosslink the polyhydroxylated swellable polymer.

In various examples, the polyhydroxylated swellable polymer can include a variety of polymers that are water absorbent and that include multiple hydroxyl groups. Specifically, “polyhydroxylated” can refer to polymers that include two or more hydroxyl groups per polymer strand. The hydroxyl groups can allow the crosslinker in the crosslinking agent to crosslink different polymer strands together. In various examples, the polymer can include any number of hydroxyl groups provided that there are two or more per strand. In certain examples, the polymer can have from 2 to about 20,000 hydroxyl groups per polymer strand.

As used herein, “swellable” refers to polymers that can absorb water. Accordingly, the swellable polymers can be sufficiently hydrophilic that the dry polymer can absorb water. Additionally, swellable polymers can have or form a polymer network that can absorb and hold water without become entirely dissolved by the water. In some examples, such a polymer network can be formed by crosslinking individual polymer strands. In some examples, the polymer can begin as a water-soluble polymer and the polymer can be crosslinked by the crosslinking agent during three-dimensional printing. Accordingly, swellable polymers as described herein can be non-crosslinked and water-soluble at the beginning of the three-dimensional printing process. The polymer can become crosslinked when the crosslinking agent is applied and this can allow the crosslinked polymer to hold water without the crosslinked structure dissolving in the water. In other examples, the swellable polymer can have some degree of crosslinking to begin with. For example, the polymer can be lightly crosslinked or partially crosslinked. Then, when the crosslinking agent is applied to the polymer, additional crosslinking can form so that the individual polymer particles are crosslinked together to form a larger crosslinked structure. As mentioned above, one characteristic of hydrogels is the ability to absorb and hold water without the polymer structure being dissolved in the water.

Non-limiting examples of polyhydroxylated swellable polymers can include polyvinyl alcohol, cellulose, gelatin, alginate, chitosan, poly(2-hydroxyethyl acrylate), poly(2-hydroxyethyl methacrylate), poly(acrylic acid), poly(methacrylic acid), poly(N,N-dimethylacrylamide), poly(N,N-diethylacrylamide), poly(N-isopropylacrylamide), and combinations thereof. In some examples, the particulate build material can include one of these polyhydroxylated swellable polymers or a combination of multiple such polymers. In other examples, the particulate build material can include a combination of polyhydroxylated swellable polymer and an additional polymer. For example, individual particles of the particulate build material can be a blend of polyhydroxylated swellable polymer and an addition polymer. In certain examples, the individual particles can include from about 90 wt % to 100 wt % polyhydroxylated swellable polymer.

The polyhydroxylated swellable polymer can have any suitable molecular weight, provided that the polymer includes polymer strands having multiple hydroxyl groups. In certain examples, the polyhydroxylated swellable polymer can have a weight average molecular weight from about 1,000 Mw to about 500,000 Mw. In other examples, the molecular weight can be from about 10,000 Mw to about 300,000 Mw or from about 20,000 Mw to about 200,000 Mw. In certain examples, the polyhydroxylated swellable polymer can be polyvinyl alcohol having a molecular weight from about 1,000 Mw to about 500,000 Mw, or from about 10,000 Mw to about 300,000 Mw, or from about 20,000 Mw to about 200,000 Mw.

The particulate build material can include polymer particles having a variety of shapes, such as substantially spherical particles or irregularly-shaped particles. In some examples, the polymer particles can be capable of being formed into three-dimensional printed objects with a resolution of about 20 μm to about 1000 μm, about 30 μm to about 800 μm, or about 40 μm to about 600 μm. As used herein, “resolution” refers to the size of the smallest feature that can be formed on a three-dimensional printed object. The particulate build material can form layers from about 20 μm to about 600 μm thick, allowing the fused layers of the printed part to have roughly the same thickness. In some examples, the layer thickness can also change when the crosslinking agent is applied to the particulate build material because the polymer of the particulate build material can absorb water and swell to an increased volume. In some examples, the overall resolution in the z-axis (i.e., depth) direction, based on the layer height of the dry polymer particles and/or the layer height when the polymer particles absorb water, can be about 20 μm to about 600 μm. The particulate build material can also have a sufficiently small particle size and sufficiently regular particle shape to provide about 20 μm to about 600 μm resolution along the x-axis and y-axis (i.e., the axes parallel to the top surface of the powder bed). For example, the particulate build material can have an average particle size from about 20 μm to about 600 μm. In other examples, the average particle size can be from about 20 μm to about 500 μm. Other resolutions along these axes can be from about 30 μm to about 400 μm or from 40 μm to about 300 μm. In further examples, the particulate build material can have a D50 particle size from about 20 μm to about 600 μm, or from about 20 μm to about 500 μm, or from about 100 μm to about 300 μm. Additionally, the particulate build material can have a D90 particle size from about 100 μm to about 800 μm, or from about 200 μm to about 600 μm, or from about 300 μm to about 500 μm, in some examples. The D50 particle size is defined as the diameter threshold at which 50% of the particles have a diameter below the threshold. Similarly, D90 particle size is defined as the diameter threshold at which 90% of the particles have a diameter below the threshold.

The particulate build material can also in some cases include a filler. The filler can include inorganic particles such as alumina, silica, fibers, carbon nanotubes, or combinations thereof. When the swellable polymer particles become crosslinked together during three-dimensional printing, the filler particles can become embedded in the crosslinked polymer network, forming a composite material. In some examples, the filler can include a free-flow agent, anti-caking agent, or the like. Such agents can prevent packing of the powder particles, coat the powder particles and smooth edges to reduce inter-particle friction, and/or absorb moisture. In some examples, a weight ratio of swellable polymer particles to filler particles can be from about 1,000:1 to about 90:10.

Turning now to the crosslinking agent, in some examples the crosslinking agent can include water and a crosslinker that is reactive with hydroxyl groups of the polyhydroxylated swellable polymer to crosslink the polyhydroxylated swellable polymer. In certain examples, the crosslinker can form hydrogen bonds with the hydroxyl groups of the polyhydroxylated swellable polymer. Thus, “reactive with” includes the capability of forming hydrogen bonds with the hydroxyl groups. In further examples, the crosslinker may react with the hydroxyl groups in other ways, such as by forming covalent bonds, ionic crosslinking, and so on.

A variety of crosslinker compounds can be suitable for crosslinking polyhydroxylated polymers. In certain examples, the crosslinker used in the crosslinker agent can be boric acid, citric acid, succinic acid, cationic calcium, cationic barium, or a combination thereof. These examples can also include salt forms of the crosslinker compounds, such as halide salts of acid crosslinkers and cationic ionic crosslinkers. For example, sodium tetraborate can be an alternative compound for boric acid. Other salt forms of crosslinker compounds can also be used. Some crosslinker compounds can be water-soluble. These crosslinkers can be dissolved in the crosslinking agent. In further examples, the crosslinking agent can include a liquid vehicle that includes water and an organic solvent. In some such examples, the crosslinker compound can be soluble in the liquid vehicle of the crosslinking agent. In alternative examples, crosslinkers can be dispersed in the crosslinking agent if the crosslinkers are not soluble.

As used herein, “water-soluble” refers to materials that can be dissolved in water at a concentration from about 5 wt % to about 99 wt % of the dissolved material with respect to the entire weight of the solution. The solution of a water-soluble material can be fully transparent without any phase separation. Materials that are not water-soluble can be referred to as “water-insoluble.”

The crosslinker concentration in the crosslinking agent can be adjusted to provide a suitable degree of crosslinking in the three-dimensional printed hydrogel. In some examples, the concentration can also be within a range that provides good jettability when the crosslinking agent is jetted from fluid ejectors during three-dimensional printing. In certain examples, the concentration of crosslinker in the crosslinking agent can be from about 0.1 wt % to about 50 wt % based on the total weight of the crosslinking agent. In further examples, the concentration can be from about 0.5 wt % to about 25 wt % or from about 1 wt % to about 20 wt %. Using a higher concentration of crosslinker can result in a relatively higher degree of crosslinking in the three-dimensional printed hydrogel. This can affect the properties of the hydrogel. For example, hydrogels with a higher degree of crosslinking can have greater mechanical strength and can be more rigid. Hydrogels with a lower degree of crosslinking can be weaker and more flexible.

In a particular example, the crosslinker can include boric acid or a borate salt, and the polyhydroxylated swellable polymer of the particulate build material can include polyvinyl alcohol. In a particular example, the crosslinker can be sodium tetraborate. When dissolved in the crosslinking agent, the sodium tetraborate can from tetrahedral borate ions, which can crosslink polyvinyl alcohol by forming hydrogen bonds with hydroxyl groups of the polyvinyl alcohol. A mechanism for crosslinking polyvinyl alcohol using tetrahedral borate ions is shown in FIG. 2. In this FIG., an asterisk (*) is shown illustrating that this is a portion of the polyvinyl alcohol polymer that can extend in either or both directions until terminated, resulting in a polymer with a weight average molecular weight set forth previously, e.g., from about 1,000 Mw. To about 500,000 Mw. The crosslinking can also be reversed, in some examples, by exposing the polymer to an acidic pH. This degradation process is also shown in FIG. 2.

In some examples, the crosslinking agent can be jetted onto the particulate build material using a fluid jetting device, such as inkjet printing architecture. Accordingly, in some examples, the crosslinking agent can be formulated to give the crosslinking agent good jetting performance. Ingredients that can be included in the crosslinking agent to provide good jetting performance can include a liquid vehicle. Thermal jetting can function by heating the crosslinking agent to form a vapor bubble that displaces fluid around the bubble, and thereby forces a droplet of fluid out of a jet nozzle. Thus, in some examples the liquid vehicle can include a sufficient amount of an evaporating liquid that can form vapor bubbles when heated. The evaporating liquid can be a solvent such as water, an alcohol, an ether, or a combination thereof.

In some examples, the liquid vehicle formulation can include a co-solvent or co-solvents present in total at from about 1 wt % to about 50 wt %, depending on the jetting architecture. Further, a non-ionic, cationic, and/or anionic surfactant can be present, ranging from about 0.01 wt % to about 5 wt %. In one example, the surfactant can be present in an amount from about 1 wt % to about 5 wt %. The liquid vehicle can include dispersants in an amount from about 0.5 wt % to about 3 wt %. The balance of the formulation can be purified water, and/or other vehicle components such as biocides, viscosity modifiers, materials for pH adjustment, sequestering agents, preservatives, and the like. In one example, the liquid vehicle can be predominantly water.

Classes of co-solvents that can be used can include organic co-solvents including aliphatic alcohols, aromatic alcohols, diols, glycols, glycol ethers, polyglycol ethers, caprolactams, formamides, acetamides, and long chain alcohols. Examples of such compounds include 1-aliphatic alcohols, secondary aliphatic alcohols, 1,2-alcohols, 1,3-alcohols, 1,5-alcohols, propylene glycol, ethylene glycol alkyl ethers, propylene glycol alkyl ethers, higher homologs (C₆-C₁₂) of polyethylene glycol alkyl ethers, N-alkyl caprolactams, unsubstituted caprolactams, both substituted and unsubstituted formamides, both substituted and unsubstituted acetamides, and the like. Specific examples of solvents that can be used include, but are not limited to, 2-pyrrolidinone, N-methylpyrrolidone, 2-hydroxyethyl-2-pyrrolidone, 2-methyl-1,3-propanediol, tetraethylene glycol, 1,6-hexanediol, 1,5-hexanediol and 1,5-pentanediol.

Regarding the surfactant that may be present, a surfactant or surfactants can be used, such as alkyl polyethylene oxides, alkyl phenyl polyethylene oxides, polyethylene oxide block copolymers, acetylenic polyethylene oxides, polyethylene oxide (di)esters, polyethylene oxide amines, protonated polyethylene oxide amines, protonated polyethylene oxide amides, dimethicone copolyols, substituted amine oxides, and the like. The amount of surfactant added to the crosslinking agent may range from about 0.01 wt % to about 20 wt %. Suitable surfactants can include, but are not limited to, liponic esters such as TERGITOL™ 15-S-12, TERGITOL™ 15-S-7 available from Dow Chemical Company (Michigan), LEG-1 and LEG-7; TRITON™ X-100; TRITON™ X-405 available from Dow Chemical Company (Michigan); and sodium dodecylsulfate.

Various other additives can be employed to enhance certain properties of the crosslinking agent for specific applications. Examples of these additives are those added to inhibit the growth of harmful microorganisms. These additives may be biocides, fungicides, and other microbial agents, which can be used in various formulations. Examples of suitable microbial agents include, but are not limited to, NUOSEPT® (Nudex, Inc., New Jersey), UCARCIDE™ (Union carbide Corp., Texas), VANCIDE® (R.T. Vanderbilt Co., Connecticut), PROXEL® (ICI Americas, New Jersey), and combinations thereof.

Sequestering agents, such as EDTA (ethylene diamine tetra acetic acid), may be included to eliminate the deleterious effects of heavy metal impurities, and buffer solutions may be used to control the pH of the fluid. From about 0.01 wt % to about 2 wt %, for example, can be used. Viscosity modifiers and buffers may also be present, as well as other additives to modify properties of the fluid as desired. Such additives can be present at from about 0.01 wt % to about 20 wt %.

In some examples, the crosslinking agent can include a colorant if colored hydrogel is desired. Many of the polyhydroxylated swellable polymers suitable for hydrogel three-dimensional printing can be white or colorless.

Accordingly, vivid colors can be obtained by using a colored crosslinking agent during three-dimensional printing. Colorants can include dyes and/or pigments. In certain examples, the colorant can include water soluble dyes such as cyan, magenta, yellow, black, or other colored dyes. Combinations of these dyes can also be used. In alternative examples, the crosslinking agent can be formulated without a colorant so that the crosslinking agent is colorless. Such crosslinking agents can be used to make colorless hydrogels. In further examples, the hydrogel three-dimensional printing kits can include addition fluid agents. For example, separate coloring agents can be used to color the three-dimensional printed hydrogel.

In many examples, the crosslinking agent can be devoid of a polymeric binder or substantially devoid of a polymeric binder. Unlike two-dimensional printing inks, which often include a polymeric binder to bind colorants to a print substrate, the crosslinking agents described herein can be designed for three-dimensional printing. In particular, the crosslinking agents can be designed to apply to swellable polymer build material. Thus, the crosslinking agents can function well in the three-dimensional printing process without any polymeric binder in the crosslinking agent. In some cases, certain polymers may be present in the crosslinking agent such as, for example, polymeric dispersants or polymers that perform other functions. However, in some examples the crosslinking agent can be devoid of a polymeric binder and any other polymers present in the agent can be present in minimal amounts, such as less than about 1 wt %, or less than about 0.5 wt %, or less than about 0.1 wt %.

In certain examples, the crosslinking agent can include from about 0.1 wt % to about 50 wt % crosslinker, from about 1 wt % to about 50 wt % organic solvent, from about 0.1 wt % to about 20 wt % surfactant, and from about 50 wt % to about 98 wt % water. In further examples, the crosslinking agent can consist of a crosslinker and a liquid vehicle. In certain examples, the liquid vehicle can consist of water, an organic solvent, and a surfactant. In still further examples, the liquid vehicle can consist of water, water and an organic solvent, or water and a surfactant.

### Methods of Three-Dimensional Printing Hydrogels

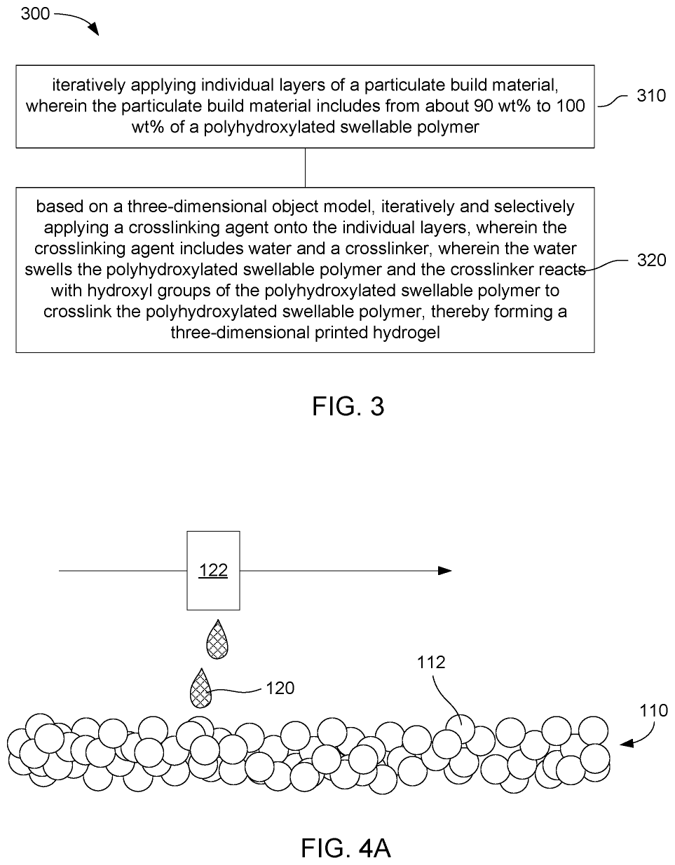

The present disclosure also describes methods of three-dimensional printing hydrogels. The materials described above in the hydrogel three-dimensional printing kits can be used in these methods. FIG. 3 shows a flowchart illustrating one example method 300 of three-dimensional printing a hydrogel. The method includes iteratively applying 310 individual layers of a particulate build material, wherein the particulate build material includes from about 90 wt % to 100 wt % of a polyhydroxylated swellable polymer. The method can further include, based on a three-dimensional object model, iteratively and selectively applying 320 a crosslinking agent onto the individual layers, wherein the crosslinking agent includes water and a crosslinker, wherein the water swells the polyhydroxylated swellable polymer and the crosslinker reacts with hydroxyl groups of the polyhydroxylated swellable polymer to crosslink the polyhydroxylated swellable polymer, thereby forming a three-dimensional printed hydrogel.

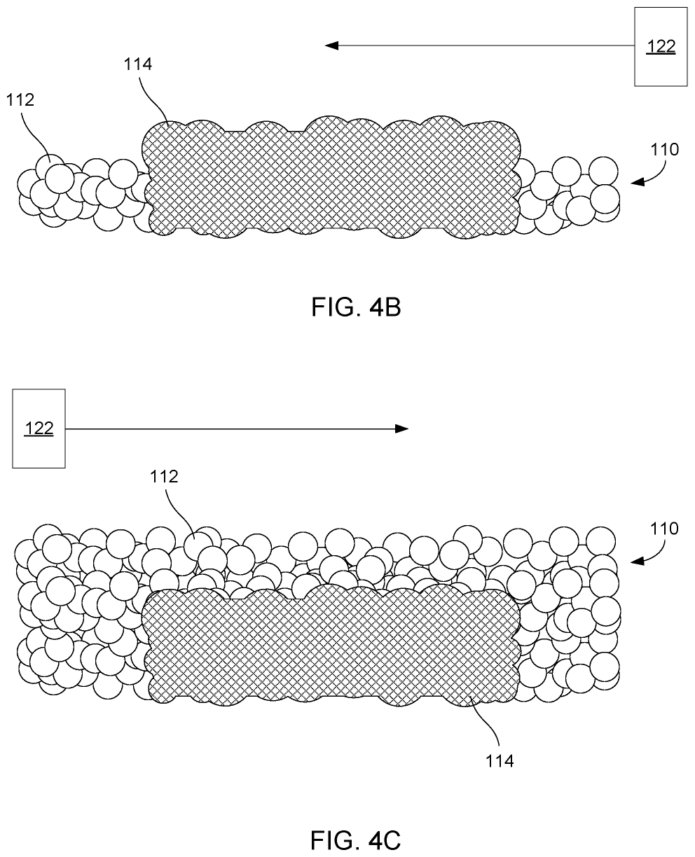

To further illustrate methods of three-dimensional printing hydrogels, FIGS. 4A-4C show one example method of three-dimensional printing a hydrogel using the hydrogel three-dimensional printing kits described above. In FIG. 4A, a crosslinking agent 120 is jetted onto a layer of particulate build material 110 made up of particles of a polyhydroxylated swellable polymer 112. The crosslinking agent is jetted from a crosslinking agent ejector 122. The crosslinking agent ejector can move across the layer of particulate build material to selectively jet the crosslinking agent on areas that are to be crosslinked to become part of the final hydrogel.

FIG. 4B shows the layer of particulate build material 110 after the crosslinking agent has been jetted onto the powder bed. The crosslinking agent has been jetted in an area of the particulate build material layer that is to be crosslinked to become part of the final three-dimensional printed hydrogel. The crosslinking agent converts the dry particulate build material into a crosslinked hydrogel 114. In this example, the polymer swells in the area where the crosslinking agent was jetted due to the polymer absorbing water from the crosslinking agent. As shown in the figure, this can result in a volume increase in the area where the crosslinking agent was jetted (shown schematically as a z-axis increase in volume), where the hydrogel has a larger volume than the original volume of the layer of particulate build material. Some volume increase can also be laterally in x- and y-axis directions.

FIG. 4C shows an additional layer of particulate build material 110 spread over the top of the previous layer. The additional layer can have a sufficient layer thickness that some particulate build material covers the hydrogel formed in the previous layer. The process of jetting the crosslinking agent onto the powder bed can then be repeated to form an area of hydrogel from the additional layer of particulate build material. The individual layers of hydrogel can correspond to layers or slices of the three-dimensional object that is to be formed of hydrogel. The crosslinking agent can cause the polymer in separate layers to crosslink together so that the layers of hydrogel form a continuous crosslinked hydrogel network. This entire process can then be repeated for many more layers to build up a three-dimensional printed hydrogel object.

The crosslinking agent can be applied to the particulate build material using a variety of methods. In some examples, the crosslinking agent can be jetted onto the build material using a fluid jet print head. The amount of crosslinking agent that is applied is calibrated based on the concentration of crosslinker in the crosslinking agent, the desired degree of crosslinking for the three-dimensional printed hydrogel, and the desired water content for the hydrogel. When the crosslinking agent is jetted onto the build material using a fluid ejector, the amount of crosslinking agent applied can be controlled by ejecting a particular number of droplets of the crosslinking agent onto a particular area of the powder bed. This can be referred to as the contone level of jetting the crosslinking agent. In certain examples, the contone level can be controlled by selecting a number of droplets to print onto an area of the powder bed that is 1/600^{th} of an inch (42 μm) by 1/600^{th} of an inch (42 μm). The size of the droplets can be known in some examples. In certain examples, the droplet size can be from about 1 ng to 50 ng, or from about 5 ng to about 25 ng, or from about 6 ng to about 15 ng. In a particular example, the droplet size can be about 9 ng. The number of droplets printed onto the area of the powder bed can be from about 50 to about 100. The total amount of the crosslinking agent applied to an area of the powder bed can affect the water content in the three-dimensional printed hydrogel. In certain examples, the contone level can be selected so that the water content of the hydrogel is from about 50 wt % to about 95 wt % based on the total weight of the hydrogel. In further examples, the water content can be from about 70 wt % to about 95 wt % or from about 80 wt % to about 95 wt %.

In some examples, the temperature of the powder bed can be controlled during three-dimensional printing. However, in other examples, the three-dimensional printing can be performed at ambient temperature without temperature control. The crosslinking agent can be applied to the polyhydroxylated swellable polymer at or near room temperature to form the hydrogel. In certain examples, the particulate build material can be at a temperature from about 0° C. to about 75° C. during three-dimensional printing of the hydrogel. In further examples, the particulate build material can be at a temperature from about 20° C. to about 50° C. or from about 30° C. to about 40° C. In another example, the particulate build material can be at a temperature of about 37° C. The crosslinking agent can also be maintained at a desired temperature so that the temperature of the particulate build material can be maintained during printing. In cases where the particulate build material is heated during three-dimensional printing, the heating can be accomplished with a heating lamp or lamps, an oven, a heated support bed, other types of heaters, or combinations thereof. In some examples, the entire powder bed can be heated to a substantially uniform temperature.

The three-dimensional printed hydrogel object can be formed to have a shape of a three-dimensional object model. Three-dimensional object models can in some examples be created using computer aided design (CAD) software. Three-dimensional object models can be stored in any suitable file format. In some examples, a three-dimensional printed hydrogel object as described herein can be based on a single three-dimensional object model. The crosslinking agent can be applied to areas of the particulate build material that correspond to layers or slices of the three-dimensional object model. Other information may also be included in three-dimensional object models, such as structures to be formed of additional different materials or color data for printing the object with various colors at different locations on the object. The three-dimensional object model may also include features or materials specifically related to jetting fluids on layers of powder bed material, such as the desired amount of fluid to be applied to a given area. This information may be in the form of a contone level or droplet saturation, for example, which can instruct a three-dimensional printing system to jet a certain number of droplets of fluid into a specific area. This can allow the three-dimensional printing system to finely control the amount of fluid agent applied to the particulate build material. All this information can be contained in a single three-dimensional object file or a combination of multiple files. The three-dimensional printed hydrogel object can be made based on the three-dimensional object model. As used herein, “based on the three-dimensional object model” can refer to printing using a single three-dimensional object model file or a combination of multiple three-dimensional object model files that together define the object. In certain examples, software can be used to convert a three-dimensional object model to instructions for a three-dimensional printer to form the object by building up individual layers of build material.

In an example of the three-dimensional printing process, a thin layer of particulate build material can be spread on a bed to form a powder bed. At the beginning of the process, the powder bed can be empty because no build material particles have been spread at that point. For the first layer, the particles can be spread onto an empty build platform. The build platform can be a flat surface made of a material such as a metal, glass, or plastic. Thus, “applying individual layers of a particulate build material” includes spreading particulate build material onto the empty build platform to provide a first layer, or subsequent layers can be applied to a powder bed of particulate build material (where some of the powder bed is part of or becomes part of the three-dimensional object being form). In other examples, a number of initial layers of particulate build material can be spread before the printing begins. These “blank” layers of particulate build material can in some examples number from about 1 to about 500, from about 10 to about 200, or from about 10 to about 100. In some cases, spreading multiple layers of powder before beginning the print can increase temperature uniformity of the three-dimensional printed hydrogel object. A fluid jet printing head, such as an inkjet print head, can then be used to print a crosslinking agent over portions of the powder bed corresponding to a thin layer of the three-dimensional printed hydrogel object. The crosslinking agent can include a crosslinker to crosslink the polymer in the particulate build material and water to be absorbed by the crosslinked polymer. This can form the first layer of hydrogel. After the first layer is formed, a new thin layer of particulate build material can be spread over the powder bed and the process can be repeated to form additional layers until a complete three-dimensional hydrogel object is printed. Thus, “applying individual layers of a particulate build material” also includes spreading layers of particulate build material over the loose particles and crosslinked hydrogel layers beneath the new layer of particulate build material.

When the three-dimensional printed hydrogel object is complete, the object can be removed from the powder bed. In certain examples, any loose particulate build material that remains in the powder bed can be recycled and used for future three-dimensional printing. In some cases, particles of the particulate build material can cling to the surfaces of the three-dimensional printed hydrogel object. These particles can be loosely adhered to the object without being crosslinked. In some examples, the adhered particles can be removed from the hydrogel object by brushing, blowing with compressed air, rinsing with water, or another method. In certain examples, the particulate build material can be a water-soluble polymer. Rinsing the hydrogel object with water can easily remove the adhered particles, as the particles can dissolve in the rinse water while the crosslinked hydrogel does not dissolve.

### Hydrogel Three-Dimensional Printing Systems

The present disclosure also extends to hydrogel three-dimensional printing systems. The systems can include the particulate build material and the crosslinking agent described above. The systems can also include a fluid applicator to apply the crosslinking agent to the particulate build material. In certain examples, the fluid applicator can be fluidly coupled or coupleable to a crosslinking agent. The fluid applicator can also be directable to iteratively apply the crosslinking agent to layers of the particulate build material. The crosslinking agent can include water to swell the polyhydroxylated swellable polymer in the particulate build material. The crosslinking agent can also include a crosslinker that is reactive to crosslink hydroxyl groups of the polyhydroxylated swellable polymer.

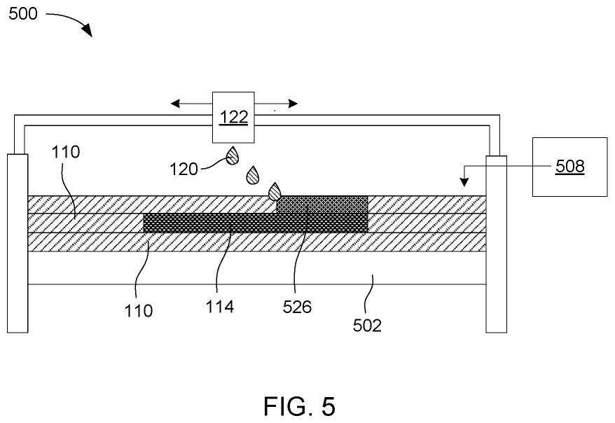

FIG. 5 shows an example hydrogel three-dimensional printing system 500 in accordance with the present disclosure. The system includes a build platform 502. Particulate build material 110 can be deposited onto the build platform or a subsequently applied layer of particulate build material (or the powder bed) by a build material applicator 508 where the particulate build material can be flattened or smoothed, such as by a mechanical roller or other flattening technique. The crosslinking agent 120 can then be applied to the layer by a crosslinking agent ejector 122. The area 526 where the crosslinking agent is applied can correspond to a layer or slice of a three-dimensional object model. FIG. 5 shows a first layer of hydrogel 114 that has already formed, with an additional layer of particulate build material spread over the top, and the system is in the process of applying the crosslinking agent to the additional layer to form another layer of hydrogel. At the area where the crosslinking agent is applied, this can swell and become a second layer of hydrogel, and so forth.

As used herein, “applying individual layers of particulate build material” can include applying the first layer of particulate build material that is applied directly to an empty support bed. The “support bed” can refer to the build platform, as shown in FIG. 5, for example. Additionally, in some examples, a layer or multiple layers of particulate build material can be laid on the support bed without jetting any crosslinking agent onto the layers. Accordingly, “applying individual layers of a particulate build material” can include applying a layer of particulate build material onto the initial layer or layers that may be applied without any crosslinking agent, or includes applying to subsequent layers, when a layer or slice of the three-dimensional printed hydrogel object has already been formed in the layer below.

In further examples, the system can include a temperature control device such as a heater and/or temperature sensors for feedback. As mentioned above, the three-dimensional printing processes described herein can be performed at relatively low temperatures, such as from about 0° C. to about 75° C. In certain examples, the three-dimensional printing process can be performed at or near body temperature or another temperature that can be suitable for living cells. In some examples, the system can include a heat lamp or other heater over the powder bed. In other examples, a heater or multiple heaters can be positioned on a side or sides of the powder bed, or a combination of these locations. In some examples, the support bed can include an additional integrated heater to heat the powder bed from below to maintain a more uniform temperature in the powder bed.

### Definitions

It is noted that, as used in this specification and the appended claims, the singular forms “a,” “an,” and “the” include plural referents unless the context clearly dictates otherwise.

As used herein, “colorant” can include dyes and/or pigments.

As used herein, “dye” refers to compounds or molecules that absorb electromagnetic radiation or certain wavelengths thereof. Dyes can impart a visible color to an ink if the dyes absorb wavelengths in the visible spectrum.

As used herein, “pigment” can include pigment colorants, magnetic particles, aluminas, silicas, and/or other ceramics, organo-metallics or other opaque particles, whether or not such particulates impart color. Thus, though the present description mentions pigment colorants, the term “pigment” can be used to describe pigment colorants and also other pigments such as organometallics, ferrites, ceramics, etc. In one specific aspect, however, the pigment is a pigment colorant.

As used herein, “ink jetting” or “jetting” refers to compositions that are ejected from jetting architecture, such as ink-jet architecture. Ink-jet architecture can include thermal or piezo architecture. Additionally, such architecture can be configured to print varying drop sizes such as less than 10 picoliters, less than 20 picoliters, less than 30 picoliters, less than 40 picoliters, less than 50 picoliters, etc.

As used herein, “average particle size” refers to a number average of the diameter of the particles for spherical particles, or a number average of the volume equivalent sphere diameter for non-spherical particles. The volume equivalent sphere diameter is the diameter of a sphere having the same volume as the particle. Average particle size can be measured using a particle analyzer such as the MASTERSIZER™ 3000 available from Malvern Panalytical (United Kingdom). The particle analyzer can measure particle size using laser diffraction.

A laser beam can pass through a sample of particles and the angular variation in intensity of light scattered by the particles can be measured. Larger particles scatter light at smaller angles, while small particles scatter light at larger angles. The particle analyzer can then analyze the angular scattering data to calculate the size of the particles using the Mie theory of light scattering. The particle size can be reported as a volume equivalent sphere diameter.

As used herein, the term “substantial” or “substantially” when used in reference to a quantity or amount of a material, or a specific characteristic thereof, refers to an amount that is sufficient to provide an effect that the material or characteristic was intended to provide. The exact degree of deviation allowable may in some cases depend on the specific context. When using the term “substantial” or “substantially” in the negative, e.g., substantially devoid of a material, what is meant is from none of that material is present, or at most, trace amounts could be present at a concentration that would not impact the function or properties of the composition as a whole.

As used herein, the term “about” is used to provide flexibility to a numerical range endpoint by providing that a given value may be “a little above” or “a little below” the endpoint. The degree of flexibility of this term can be dictated by the particular variable and determined based on the associated description herein.

As used herein, a plurality of items, structural elements, compositional elements, and/or materials may be presented in a common list for convenience. However, these lists should be construed as though members of the list are individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include the numerical values explicitly recited as the limits of the range, and also to include individual numerical values or sub-ranges encompassed within that range as if numerical values and sub-ranges are explicitly recited. As an illustration, a numerical range of “about 1 wt % to about 5 wt %” should be interpreted to include the explicitly recited values of about 1 wt % to about 5 wt %, and also to include individual values and sub-ranges within the indicated range. Thus, included in this numerical range are individual values such as 2, 3.5, and 4 and sub-ranges such as from 1-3, from 2-4, and from 3-5, etc.

This same principle applies to ranges reciting a single numerical value. Furthermore, such an interpretation should apply regardless of the breadth of the range or the characteristics being described.

### EXAMPLES

The following illustrates examples of the present disclosure. However, it is to be understood that the following are merely illustrative of the application of the principles of the present disclosure. Numerous modifications and alternative devices, methods, and systems may be devised without departing from the spirit and scope of the present disclosure. The appended claims are intended to cover such modifications and arrangements.

### Example 1

### Crosslinking Agent Formulation

A sample crosslinking agent was prepared by mixing the following ingredients. Sodium tetraborate was used as the crosslinker compound. The sodium tetraborate was included in an amount of 10 wt %. The crosslinking agent also include an organic co-solvent in an amount of 10 wt %, a surfactant in an amount of 0.8 wt %, and deionized water in an amount of 79.2 wt %.

The sample crosslinking agent was tested for jettability by loading the crosslinking agent into a two-dimensional inkjet printer. A cyan dye was added to the crosslinking agent to make the agent visible when printed. A test pattern was printed to evaluate nozzle health and decap of the inkjet printer when printing the crosslinking agent. The results showed excellent nozzle health and decap up to 18 seconds.

### Example 2

### Three-Dimensional Printing Evaluation

The sample crosslinking agent of Example 1 was loaded in a test three-dimensional printer that included a powder bed and an inkjet printhead for jetting the crosslinking agent onto the powder bed. The particulate build material used in the powder bed was a dry non-crosslinked polyvinyl alcohol powder. The layer height was set at 400 μm, meaning that when a fresh layer of particulate build material was spread on the powder bed, the upper surface of the layer was 400 μm higher than the previous layer. The amount of crosslinking agent jetted onto the powder bed was varied between 50 and 100 droplets (having a weight of 9 ng per droplet) per square of 1/600^{th} inch by 1/6004^{th} inch. This amount of crosslinking agent corresponded to a layer of liquid having a depth of 200-400 μm deposited onto the individual layers of particulate build material.

It was found that when the crosslinking agent was jetted onto a layer of particulate build material, the polyvinyl alcohol absorbed the water in the crosslinking agent and swelled to a greater volume. This caused the layer height to increase. When the next layer of dry particulate build material was spread over the powder bed, there was less space over the swelled area, so that the amount of powder added over that area in particular was less than 400 μm deep. In some cases, there was space for about 40 μm of additional particulate build material over the top of the swelled area.

A series of sample hydrogel objects was printed using the test three-dimensional printer. The temperature of the powder bed was maintained at less than 37° C. during printing. The crosslinking agent was tinted with cyan dye, which gave the hydrogel a bright blue color. The hydrogel objects were successfully printed and removed from the powder bed. White particles of dry polyvinyl alcohol powder were adhered to the surfaces of the hydrogel objects. To remove these particles, the hydrogel objects were submerged in water for several minutes. The un-crosslinked polyvinyl alcohol particles dissolved in the water, leaving the bright blue hydrogel objects.

The hydrogel objects were examined under magnification and this showed a relatively isotropic surface of the hydrogel. There were no visible layer lines between the individual layers that were formed during three-dimensional printing. These results show that hydrogel objects can be successfully printed using the systems and methods described herein. The hydrogels can be printed at relatively low temperatures and the hydrogels can have a relatively high water content.

What is claimed is:

1. A hydrogel three-dimensional printing kit comprising:
a particulate build material comprising from about 90 wt % to 100 wt % of a polyhydroxylated swellable polymer; and
a crosslinking agent comprising water and a crosslinker that is reactive with hydroxyl groups of the polyhydroxylated swellable polymer to crosslink the polyhydroxylated swellable polymer.

2. The hydrogel three-dimensional printing kit of claim 1, wherein the polyhydroxylated swellable polymer comprises polyvinyl alcohol, cellulose, gelatin, alginate, chitosan, poly(2-hydroxyethyl acrylate), poly(2-hydroxyethyl methacrylate), poly(acrylic acid), poly(methacrylic acid), poly(N,N-dimethylacrylamide), poly(N,N-diethylacrylamide), poly(N-isopropylacrylamide), or a combination thereof.

3. The hydrogel three-dimensional printing kit of claim 1, wherein the polyhydroxylated swellable polymer is polyvinyl alcohol having a weight average molecular weight from about 1,000 Mw to about 500,000 Mw.

4. The hydrogel three-dimensional printing kit of claim 1, wherein the crosslinker is reactive to form hydrogen bonds with the hydroxyl groups of the polyhydroxylated swellable polymer.

5. The hydrogel three-dimensional printing kit of claim 1, wherein the crosslinker comprises boric acid, citric acid, succinic acid, cationic calcium, cationic barium, or a combination thereof.

6. The hydrogel three-dimensional printing kit of claim 1, wherein the crosslinker is present in the crosslinking agent at a concentration from about 0.1 wt % to about 50 wt %.

7. A method of three-dimensional printing a hydrogel comprising:
iteratively applying individual layers of a particulate build material, wherein the particulate build material comprises from about 90 wt % to 100 wt % of a polyhydroxylated swellable polymer; and
based on a three-dimensional object model, iteratively and selectively applying a crosslinking agent onto the individual layers, wherein the crosslinking agent comprises water and a crosslinker, wherein the water swells the polyhydroxylated swellable polymer and the crosslinker reacts with hydroxyl groups of the polyhydroxylated swellable polymer to crosslink the polyhydroxylated swellable polymer, thereby forming a three-dimensional printed hydrogel.

8. The method of claim 7, wherein the particulate build material is at a temperature from about 0° C. to about 75° C. during three-dimensional printing of the hydrogel.

9. The method of claim 7, wherein the crosslinking agent is applied onto the individual layers at a contone level so that the three-dimensional the individual layers include from about 50 wt % to about 95 wt % water based on a total weight of individual layer and crosslinking agent applied.

10. The method of claim 7, further comprising removing the three-dimensional printed hydrogel from the powder bed and rinsing the three-dimensional printed hydrogel with water.

11. The method of claim 7, wherein the polyhydroxylated swellable polymer comprises polyvinyl alcohol, cellulose, gelatin, alginate, chitosan, poly(2-hydroxyethyl acrylate), poly(2-hydroxyethyl methacrylate), poly(acrylic acid), poly(methacrylic acid), poly(N,N-dimethylacrylamide), poly(N,N-diethylacrylamide), poly(N-isopropylacrylamide), or a combination thereof.

12. The method of claim 7, wherein the crosslinker is present in the crosslinking agent in an amount from about 0.1 wt % to about 50 wt %, and wherein the crosslinker comprises boric acid, citric acid, succinic acid, cationic calcium, cationic barium, or a combination thereof.

13. A hydrogel three-dimensional printing system comprising:
a particulate build material including from about 90 wt % to 100 wt % of a polyhydroxylated swellable polymer; and
a fluid applicator fluidly coupled or coupleable to a crosslinking agent, wherein the fluid applicator is directable to iteratively apply the crosslinking agent to layers of the particulate build material, wherein the crosslinking agent comprises water to swell the polyhydroxylated swellable polymer and a crosslinker that is reactive to crosslink hydroxyl groups of the polyhydroxylated swellable polymer.

14. The system of claim 13, wherein the polyhydroxylated swellable polymer comprises polyvinyl alcohol, cellulose, gelatin, alginate, chitosan, poly(2-hydroxyethyl acrylate), poly(2-hydroxyethyl methacrylate), poly(acrylic acid), poly(methacrylic acid), poly(N,N-dimethylacrylamide), poly(N,N-diethylacrylamide), poly(N-isopropylacrylamide), or a combination thereof.

15. The system of claim 13, wherein the crosslinker is present in the crosslinking agent in an amount from about 0.1 wt % to about 50 wt %, and wherein the crosslinker comprises boric acid, citric acid, succinic acid, cationic calcium, cationic barium, or a combination thereof.
